(19) United States
(12) Patent Application Publication
(10) Pub. No.: US 2012/0259314 A1
(43) Pub. Date: Oct. 11, 2012

(54) Apparatus and Methods for Recanalization of a Chronic Total Occlusion

(75) Inventors: Ya Guo, Santa Rosa, CA (US); Stefan Tunev, Santa Rosa, CA (US); Joseph Traina, Napa, CA (US)

(73) Assignee: Medtronic Vascular, Inc., Santa Rosa, CA (US)

(21) Appl. No.: 13/083,764

(22) Filed: Apr. 11, 2011

(51) Int. Cl. A61M 25/09; A61M 25/10

(52) U.S. Cl. 604/509; 604/510

(57) ABSTRACT

A method of recanalizing a chronic total occlusion (CTO) is disclosed. A catheter with a guidewire slidingly received therein is positioned proximally adjacent to the CTO. An occlusion weakening therapy effective to quickly soften and/or loosen the CTO is delivered to the CTO via the catheter. The occlusion weakening therapy includes at least one enzyme and a chelating agent which are selectively deliverable either together or separately depending on the type of material encountered by the guidewire. According to various methods, the enzyme(s) may be delivered if tissue is encountered, the chelating agent may be delivered if calcification is encountered, and both enzyme(s) and chelating agent(s) may be delivered if a fibrous cap is encountered. The distal end of the guidewire may be advanced into the CTO shortly after delivery of the occlusion weakening therapy and is manipulated through the CTO until the guidewire successfully crosses the CTO.

### FIELD OF THE INVENTION

The invention relates generally to intraluminal methods and devices for the treatment of a chronic total occlusion (CTO) within a body vessel.

### BACKGROUND OF THE INVENTION

Stenotic lesions may include a hard, calcified substance and/or a softer thrombus material, each of which forms on the lumen walls of a blood vessel and restricts blood flow. Intraluminal treatments such as balloon angioplasty, stent deployment, atherectomy, and thrombectomy are well known and have proven effective in the treatment of such stenotic lesions. These treatments often involve the insertion of a therapy catheter into a patient's vasculature, which may be tortuous and may have numerous stenoses of varying degrees along its length. In order to place a distal end of a catheter at the treatment site, a guidewire is typically introduced and tracked from an incision, through the vasculature, and across the lesion. Then, a balloon catheter, perhaps containing a stent at its distal end, can be tracked over the guidewire to the treatment site. Ordinarily, a distal end of the guidewire is quite flexible so that it may be rotatably steered and pushed through the bifurcations and turns of the typically irregular passageway without damaging the vessel walls.

In some instances, the extent of occlusion of the lumen is so severe that the lumen is completely or nearly completely obstructed, which may be described as a total occlusion. If such an occlusion persists for a long period of time, the lesion may be referred to as a chronic total occlusion or CTO. Furthermore, in the case of diseased blood vessels, the lining of the vessels may be characterized by the prevalence of atheromatous plaque, which may form total occlusions. The extensive plaque formation of a chronic total occlusion typically has a fibrous cap surrounding softer plaque material. This fibrous cap may present a surface that is difficult to penetrate with a conventional guidewire, and a typically flexible distal tip of the guidewire may be unable to cross the lesion.

Thus, for treatment of total occlusions, stiffer guidewires have been employed to recanalize through the total occlusion. However, due to the fibrous cap of the total occlusion, a stiffer guidewire still may not be able to cross the occlusion and may prolapse into the vessel when force is applied. As well when using a stiffer guidewire, greater care must be taken to avoid perforation of the vessel wall.

Further, even if the guidewire can penetrate the proximal cap of the total occlusion, it may not be able to completely cross the occlusion. In a CTO, there may be a distortion of the regular vascular architecture such that there may be multiple small non-functional channels throughout the occlusion rather than one central lumen for recanalization. Thus, the conventional approach of looking for a single channel in the center of the occlusion may account for many of the failures. Furthermore, these spontaneously recanalized channels may be responsible for failures due to their dead-end pathways and misdirecting of the guidewires. Once a “false” tract is created by a guidewire, subsequent attempts with different guidewires may continue to follow the same incorrect path, and it is very difficult to steer subsequent guidewires away from the false tract.

Another equally important failure mode, even after a guidewire successfully crosses a chronic total occlusion, is the inability to advance a balloon, other angioplasty device, or other intravascular device over the guidewire due to the fibrocalcific composition of the chronic total occlusion, which occur as fibrous caps mainly at the “entry” and “exit” segments of the chronic total occlusion.

### BRIEF SUMMARY OF THE INVENTION

Embodiments hereof relate to a method of recanalizing a chronic total occlusion. The method includes positioning distal ends of a catheter and a guidewire proximally adjacent to the occlusion. An occlusion weakening therapy effective to soften and/or loosen the occlusion is delivered to the occlusion via at least one lumen of a catheter. The occlusion weakening therapy includes at least a first therapeutic agent targeted to soften and/or loosen a first material or component of the occlusion and a second therapeutic agent targeted to soften and/or loosen a second material or component of the occlusion, and the first and second therapeutic agents are selectively deliverable together or separately depending on the type of material encountered in the occlusion that needs to be crossed or recanalized by the guidewire. The distal end of the guidewire is advanced into the occlusion until the distal end of the guidewire crosses the occlusion to exit distal thereof.

Another method according to an embodiment hereof includes positioning distal ends of a catheter and guidewire proximally adjacent to the occlusion and delivering an occlusion weakening therapy through the distal end of the catheter at or within the occlusion. The occlusion weakening therapy is effective to quickly soften and/or loosen the occlusion for crossing shortly after delivery thereof. The distal end of the guidewire is advanced into the occlusion after the delivery of the occlusion weakening therapy until the distal end of the guidewire is located through the occlusion at a point distal to the occlusion. In an embodiment, the guidewire may be advanced into the occlusion within a thirty minute time period required to weaken the occlusion.

### BRIEF DESCRIPTION OF DRAWINGS

The foregoing and other features and advantages of the invention will be apparent from the following description of embodiments hereof as illustrated in the accompanying drawings. The accompanying drawings, which are incorporated herein and form a part of the specification, further serve to explain the principles of the invention and to enable a person skilled in the pertinent art to make and use the invention. The drawings are not to scale.

FIG. 1 is a side view of a catheter for CTO recanalization according to an embodiment hereof.

FIG. 1A is a cross-sectional view taken along line A-A of FIG. 1.

FIG. 2 is a cross-sectional view taken along line A-A of FIG. 1 according to another embodiment hereof.

FIG. 3 is a cross-sectional view taken along line A-A of FIG. 1 according to another embodiment hereof.

FIGS. 4-8 diagrammatically illustrate the steps of a method of delivering an occlusion weakening therapy to a chronic total occlusion according to embodiments hereof.

FIG. 9 is a side view of a distal end of a catheter having an extendable needle tip according to another embodiment hereof, wherein the extendable needle tip is in an extended configuration or position.

FIG. 10 is a side view of a distal end of a catheter having an expandable suction cup according to another embodiment hereof, wherein the suction cup is in an expanded configuration.

FIG. 11 is a side view of a distal end of a catheter having an inflatable centering balloon according to another embodiment hereof, wherein the centering balloon is in an inflated configuration.

### DETAILED DESCRIPTION OF THE INVENTION

Specific embodiments of the present invention are now described with reference to the figures, wherein like reference numbers indicate identical or functionally similar elements. The terms “distal” and “proximal” are used in the following description with respect to a position or direction relative to the treating clinician. “Distal” or “distally” are a position distant from or in a direction away from the clinician. “Proximal” and “proximally” are a position near or in a direction toward the clinician.

The following detailed description is merely exemplary in nature and is not intended to limit the invention or the application and uses of the invention. Although the description of the invention is in the context of treatment of blood vessels such as the coronary and peripheral arteries, the invention may also be used in any other body passageways where it is deemed useful. Furthermore, there is no intention to be bound by any expressed or implied theory presented in the preceding technical field, background, brief summary or the following detailed description.

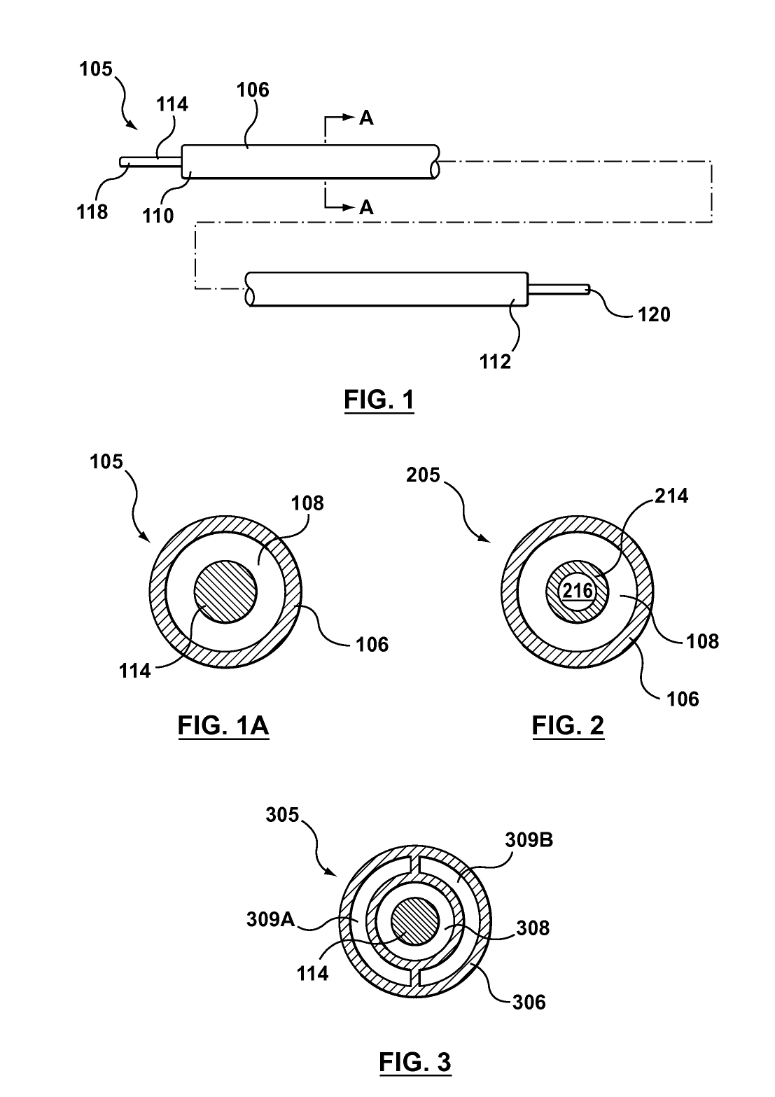

FIG. 1 is a schematic side view of a catheter 105 for CTO recanalization including a catheter shaft 106 and a guidewire 114, with FIG. 1A showing a cross-sectional view taken along line A-A in FIG. 1. Proximal ends 110, 118 of catheter shaft 106 and guidewire 114, respectively extend out of the patient and may be manipulated by a clinician, and distal ends 112, 120 of catheter shaft 106 and guidewire 114, respectively are positionable at a target location within the vasculature such as a chronic total occlusion. Catheter shaft 106 is an elongated tubular component that defines a lumen 108 for receiving guidewire 114, and may be formed of any suitable flexible polymeric material including but not limited to silicone, polyethylene terephalate (PET), nylon, polyethylene, PEBAX, or combinations of any of these, either blended or co-extruded. Optionally, a portion of catheter shaft 106 may be formed as a composite having a reinforcement material incorporated within a polymeric body in order to enhance strength, flexibility, and/or toughness. Suitable reinforcement layers include braiding, wire mesh layers, embedded axial wires, embedded helical or circumferential wires, and the like. In an embodiment, the proximal portion of catheter shaft 106 may in some instances be formed from a reinforced polymeric tube, for example, as shown and described in U.S. Pat. No. 5,827,242 to Follmer et al. which is incorporated by reference herein in its entirety. Catheter shaft 106 may have any suitable working length, for example, 550 mm-650 mm, in order to extend to a target location within the vasculature.

Guidewire 114 slidingly extends through guidewire lumen 108 of catheter shaft 106 with distal end or tip 120 of guidewire 114 extending beyond distal end 112 of catheter shaft 120 as shown in FIG. 1. In one embodiment, the outer diameter of guidewire 114 ranges between 0.014 inches and 0.020 inches. Tip 120 includes a geometry that is capable of piercing into a CTO and has the structural integrity to maintain this geometry. As shown, tip 120 may be a blunt end or alternatively may be pointed or rounded. Further, tip 120 may have a tapered frusto-conical or conical configuration as it extends distally.

Catheter 105 is utilized for delivering an occlusion weakening therapy that includes one or more active therapeutic agents to soften and/or loosen the material of a CTO. Atherosclerotic plaques vary considerably in their composition from site to site, but certain features are common to all of them. For example, plaques contain many cells which primarily are derived from cells of the vessel wall that have divided and grown into the surface layer of the blood vessel, creating a mass lesion. Plaques also contain cholesterol and cholesterol esters, commonly referred to as fat, which may lie freely in the space between the cells and in the cells themselves. A large amount of collagen is also present in the plaques, particularly advanced plaques of the type which cause clinical problems such as CTO. Additionally, human plaques contain calcium to varying degrees, hemorrhagic material including clot and grumous material composed of dead cells, and other debris. Relatively large amounts of water are also present, as is typical of all tissue. In various embodiments hereof, the occlusion weakening therapy delivered via catheter 105 includes one or more enzyme(s) and at least one chelating agent to quickly weaken the CTO, to facilitate the passing of guidewire 114 and subsequently allow for secondary interventions such as a balloon angioplasty and/or stenting. In an embodiment, the occlusion weakening therapy delivered softens or loosens the CTO within a time period of thirty minutes or less. Guidewire 114 is used to mechanically penetrate and pass through the weakened lesion during and/or shortly after administration of the occlusion weakening therapy, thereby combining mechanical force and multiple active therapeutic agents to achieve crossing of the CTO.

The particular combination of enzyme(s) and chelating agent(s) operate to quickly weaken the CTO because each therapeutic agent targets a different aspect or material of a CTO. In addition, the chelating agent enhances the activity/performance of the enzyme as compared to occlusion weakening therapy including only enzyme(s) as the active therapeutic agent. More particularly, the enzyme(s) of the occlusion weakening therapy operate to loosen or break up tissue material of a CTO. “Tissue material” as utilized herein is intended to mean connective tissue of a chronic total occlusion that may include cells primarily derived from vessel wall cells or other cells and collagen, elastin, fibronecting, laminin or other fibrous proteins, proteoglycans, hyaluronic acid, cholesterol and cholesterol esters, water, polysaccharides and/or necrotic debris, although the specific composition of atherosclerotic plaques and CTOs vary between individuals. Enzyme(s) may be selected to act on any of the above-mentioned extracellular matrix components. In one embodiment, enzyme(s) may be selected to act to degrade the collagen content of the CTO, as collagen is a predominant component of atherosclerotic plaque and is a main supportive structure of plaque of a CTO such that the plaque body collapses when the collagen degrades. Suitable examples include, but are not limited to, different proteases or elastases such as but not limited to papain, collagenase, serrapatase, or elastase which can digest proteins in the presence of extracellular matrix. In one embodiment, the one or more enzyme(s) of the occlusion weakening therapy may be selected from: matrix metalloproteinases, serine elastases, trypsin, neutral protease, chymotrypsin, aspartase, cysteinase and clostripain. Matrix metalloproteinases (MMPs) is a group of zinc-containing enzyme(s) that are responsible for degradation of several extracellular matrix (ECM) components, including collagen, fibronectin, elastin, proteoglycans and laminin. These ECM components are important components of the occluding atherosclerotic plaque. MMPs play an important role in normal embryogenesis, inflammation, wound healing and tumour invasion. These enzyme(s) are broadly classified into three general groups: collagenases, gelatinases and stromelysins. Collagenase is the initial mediator of the extracellular pathways of interstitial collagen degradation, with cleavage at a specific site in the collagen molecule, rendering it susceptible to other neutral proteases (e.g. gelatinases) in the extracellular space. In one embodiment, the enzyme containing formulation includes a matrix metalloproteinase selected from: collagenase, type 1A collagenase, gelatinases, and stromelysins. In another embodiment, the enzyme containing formulation includes collagenase, whether alone or in combination with other enzyme(s).

In addition to enhancing the performance of the enzyme(s), the chelating agent of the occlusion weakening therapy operates to soften the calcification of a CTO. Chelating agents or chelants are chemicals that bind with or “sequester” certain metal ions such as calcium (Ca²⁺). In a CTO, calcium may be loosely held in plaque deposits by an electrostatic charge which prevents the body from dissolving the plaque. The calcium of a calcified lesion binds with the chelating agent(s) of the occlusion weakening therapy, removing the metallic ion from a CTO by holding it in solution and thereby softening or dissolving the calcification of the CTO. Further, when utilized in combination with enzymes, the effects of the chelating agent(s) to soften and/or remove calcification are accelerated because enzyme(s) are simultaneously acting to loosen or break up tissue of the CTO. Non-exhaustive examples of suitable chelating agents include ethylenediaminetetraacetic acid (EDTA, a polyamino carboxylic acid), ethylene glycol tetraacetic acid (EGTA, a polyamino carboxylic acid), citric acid, and other substances that chelate with Ca+ ions.

In one embodiment shown in FIG. 1A, catheter 105 includes a single delivery lumen 108 defined by an interior surface of catheter shaft 106. Enzyme(s) and chelating agent(s) may be combined into one blended solution and delivered through delivery lumen 108 of catheter 105. Alternatively, enzyme(s) and chelating agent(s) may be delivered as separate solutions that are independently and selectively delivered through delivery lumen 108 of catheter 105. More particularly, when tissue within a CTO is encountered, a solution containing the enzyme(s) may be released through delivery lumen 108 of catheter 105. When calcification within a CTO is encountered, a solution containing chelating agent(s) may be released through delivery lumen 108 of catheter 105. If a proximal or distal fibrous cap of a CTO is encountered, a blended solution of enzyme(s) and chelating agent(s) is released to effectively weaken the fibrous cap. Thus, single delivery lumen 108 may be utilized for selective delivery of an enzyme solution, a chelating agent solution, or a blended solution of enzyme(s) and chelating agent(s).

In another embodiment shown in FIG. 2, a dual-lumen catheter 205 includes dual lumens for separately containing the enzyme(s) and chelating agent(s) for selective delivery thereof. More particularly, similar to catheter 105 described with respect to FIG. 1A above, catheter 205 includes a first delivery lumen 208 defined by an interior surface of a catheter shaft 206. Guidewire 214 is a hollow tubular member defining a second delivery lumen 216 that is slidable positionable within catheter shaft first delivery lumen 208. Accordingly, first delivery lumen 208 may be utilized for delivery of the enzyme(s) and second delivery lumen 216 may be utilized for delivery of the chelating agent(s), or vice versa. As described above, the enzyme(s) and the chelating agent(s) may each be released as necessary depending on whether tissue or calcification is encountered during advancement of guidewire 214. For example, enzyme(s) may be released through first delivery lumen 208 of catheter 205 when tissue within a CTO is encountered and chelating agents may be released through second delivery lumen 216 when calcification within a CTO is encountered. In addition, enzyme(s) and chelating agents may be simultaneously delivered through first and second delivery lumens, respectively. For example, simultaneous delivery of the enzyme(s) and chelating agent(s) may be desired if a fibrous cap of a CTO is encountered.

In another embodiment shown in FIG. 3, a multi-lumen catheter 305 includes multiple lumens for separately containing the enzyme(s) and chelating agent(s) for selective delivery thereof. More particularly, catheter 305 includes an extruded tubular catheter shaft 306 that defines first delivery lumen 308, second delivery lumen 309A, and third delivery lumen 309B. As shown in the cross-sectional view of FIG. 3, first delivery lumen 308 may have a generally circular cross-section and may accommodate a guidewire 314 therethrough. It will be understood by those of ordinary skill in the art that guidewire 314 may be a hollow guidewire similar to guidewire 214 to thereby have an additional delivery lumen if desired. Second and third delivery lumens 309A, 309B have semi-circular cross-sections and are formed within the wall of extruded tubular catheter shaft 306. Second delivery lumen 309A may be utilized for delivery of the enzyme(s) and third delivery lumen 309B may be utilized for delivery of the chelating agent(s), or vice versa. As described above, the enzyme(s) and the chelating agent(s) may each be released as necessary depending on whether tissue or calcification is encountered during advancement of guidewire 314. In addition, enzyme(s) and chelating agents may be simultaneously delivered through second and third delivery lumens 309A, 309B, respectively, or alternatively, a blended solution of enzyme(s) and chelating agent(s) may be delivered through first delivery lumen 308 between an interior surface of catheter shaft 306 and an outer surface of a guidewire 314.

Other types of catheter construction are also amendable to the present invention, such as, without limitation thereto, a catheter coaxial construction including coaxial outer and inner shafts such that a first annular lumen for delivery of occlusion weakening therapy is defined between an inner surface of the outer shaft and an outer surface of the inner shaft and a second lumen is defined by an interior surface of the inner shaft, which also serves as a guidewire lumen. As well various multi-lumen extrusion catheter shaft constructions and catheters having a rapid exchange configuration may be adapted for use herein.

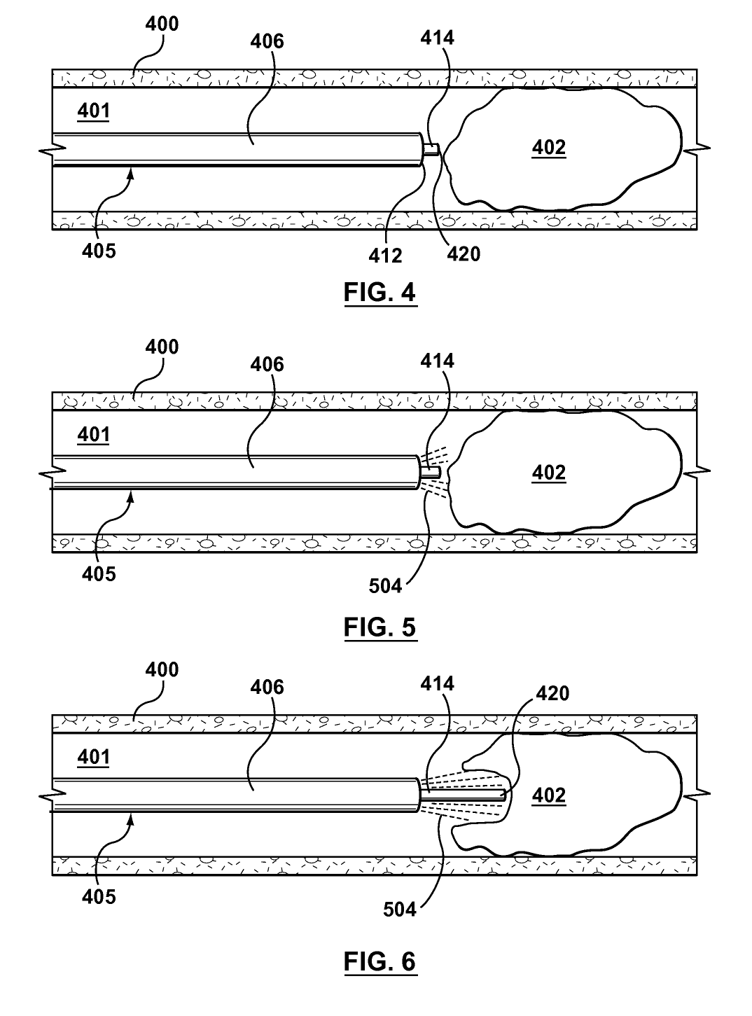

FIGS. 4-8 diagrammatically illustrate the steps of a method of delivering occlusion weakening therapy that includes one or more active agents to a chronic total occlusion 402 located within a lumen 401 of a blood vessel 400. Typically, a target vessel such as a femoral artery of a patient is punctured with a sharp hollow needle called a trocar (not shown), with ultrasound guidance if necessary, and guidewire 414 is then advanced through the lumen of the trocar. The trocar is withdrawn and a guiding catheter or sheath (not shown) is tracked over the guidewire into the vessel. A catheter 405 is then advanced through the guiding catheter and tracked over the indwelling guidewire through the vasculature. Guidewire 414 may be advanced into the target artery or vessel having occlusion 402 and catheter shaft 406 may be subsequently advanced thereover to the treatment site, or alternatively guidewire 414 and catheter shaft 406 may be simultaneously tracked to occlusion 402. Catheter 405 may be a single lumen catheter such as but not limited to catheter 105, a dual lumen catheter such as but not limited to catheter 205, or a multi-lumen catheter such as but not limited to catheter 305. As shown in FIG. 4, catheter 405 is positioned by a clinician such that a distal end 412 of catheter shaft 406 is proximally adjacent to occlusion 402 in vessel 400.

Referring to FIG. 5, occlusion weakening therapy 504 is delivered directly to occlusion 402 via one or more lumen(s) of catheter 405. As described above, occlusion weakening therapy 504 includes one or more enzyme(s) and one or more chelating agent(s). The enzyme(s) and chelating agent(s) of occlusion weakening therapy 504 may be delivered as one blended solution through a common or single delivery lumen of catheter 405. Alternatively, as described above, the enzyme(s) and chelating agent(s) of occlusion weakening therapy 504 may be selectively delivered separately, either consecutively or concurrently, through one or more delivery lumen(s) of catheter 405 as necessary during advancement of guidewire 414 depending on whether tissue or calcification is encountered. Through prior experience and expertise, the operator of catheter 405 often can tactilely distinguish, i.e., via touch or feel, what type of material, i.e., tissue or calcification, is being encountered by distal ends 412, 420 of catheter shaft 406 and guidewire 414, respectively. In another embodiment, an ultrasound transducer (not shown) may be incorporated into catheter 405 in order to determine the location of distal end 412 of catheter shaft 406 and/or distal end 120 of guidewire 414 and in order to determine what type of material is being encountered. Ultrasound technology known in the art may be utilized to distinguish between different types of material via resulting refraction waves of differing material densities. Regardless of the type of method used to identify the encountered material, the enzyme(s) and/or the chelating agent(s) are selectively delivered to occlusion 402. As previously described, enzyme(s) may be released through a lumen of catheter 405 when tissue within occlusion 402 is encountered and chelating agent(s) may be released when calcification within occlusion 402 is encountered. If occlusion 402 includes a proximal or distal fibrous cap, both enzyme(s) and the chelating agent(s) are delivered together to effectively weaken the fibrous cap. To deliver together, the enzyme(s) and chelating agent(s) may be delivered as a single, blended solution through a single or common lumen of catheter 405 or may be simultaneously released each through a separate lumen of catheter 405.

Shortly after release of occlusion weakening therapy 504, guidewire 414 is distally advanced towards and/or within occlusion 402. Thus, distal end 420 of guidewire 414 penetrates and is manipulated into occlusion 414 shortly after release of occlusion weakening therapy 504. Advantageously, occlusion weakening therapy 504 softens and/or loosens occlusion 402 shortly after administration thereof to render occlusion 402 crossable in a relative short period of time, i.e., less than thirty minutes. In one embodiment, occlusion weakening therapy 504 softens and/or loosens and renders occlusion 402 crossable in less than five minutes. Such a reduced treatment time is substantially shorter than prior art approaches to weakening a chronic total occlusion, which typically require a waiting period of one to three days between application of a weakening agent and advancement of the guidewire. The shortened treatment time may be due to the specific combination of enzyme(s) and chelating agent(s) of occlusion weakening therapy 504. More particularly, as previously described herein, in addition to being effective in the softening and/or degrading of calcification of occlusion 402, certain chelating agents such as EDTA promote or increase the enzyme's performance. In addition, the shortened treatment time is due to the relatively higher concentrations of enzyme(s) utilized in occlusion weakening therapy 504. Some known approaches for weakening a CTO typically utilize relatively diluted concentrations of enzyme(s), i.e., 300 ug/ml, due to concerns regarding higher concentrations remaining/mixing into a patient's bloodstream. In accordance with methods hereof, occlusion weakening therapy 504 utilizes higher concentrations of enzyme(s), i.e., approximately 7 mg/ml, and one or more excess enzyme removal steps to remove excess enzyme(s) from the patient to alleviate any concerns regarding the higher concentrations. As will be described in more detail herein, the enzyme removal process may include utilizing an extendable needle or cannula at distal end 420 of guidewire 414 to guide delivery of occlusion weakening therapy 504 into occlusion 402, utilizing an expandable suction cup at distal end 420 of guidewire 414 to act as a shield that prevents occlusion weakening therapy 504 from entering into the bloodstream, utilizing an inflatable balloon at distal end 420 of guidewire 414 to act as a shield that prevents occlusion weakening therapy 504 from entering into the bloodstream, and/or aspirating excess solution and debris shortly after delivery of occlusion weakening therapy 504.

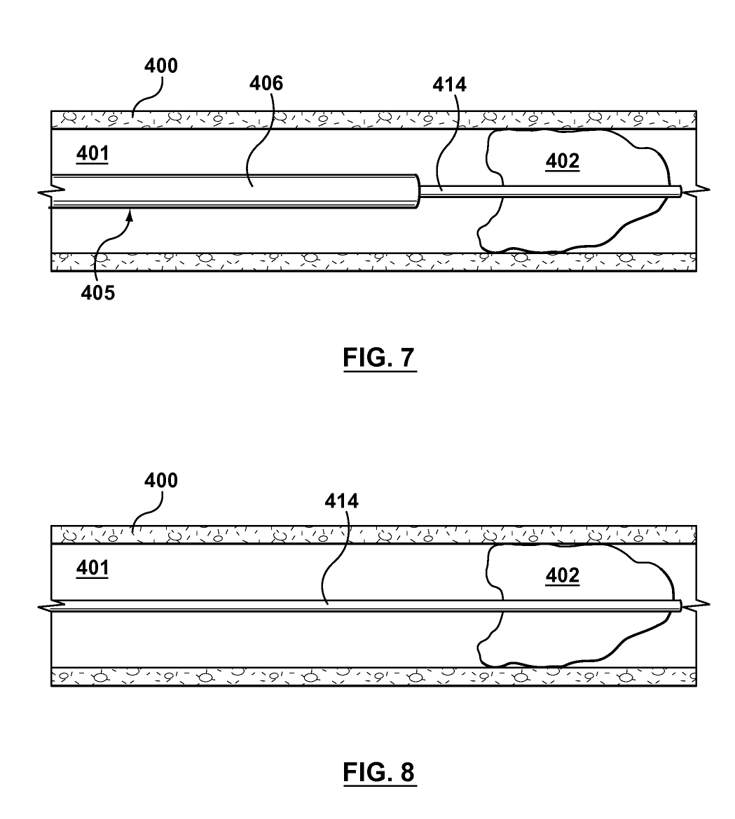

Catheter 405 is continually advanced and occlusion weakening therapy 504 released as necessary until distal end 420 of guidewire 414 is located at a point distal to occlusion 402 as shown in FIG. 7. During crossing of occlusion 402, occlusion weakening therapy 504 may be delivered on a continuous basis or only as needed. In addition, during crossing of occlusion 402, the type of occlusion weakening therapy 504 may be changed due to the type of material encountered by distal end 420 of guidewire 414. For example, in one embodiment, distal end 420 of guidewire 414 initially encounters a proximal fibrous cap, so delivered occlusion weakening therapy 504 includes both enzyme(s) and the chelating agent(s), either as separate solutions delivered concurrently or as a single blended solution, to effectively weaken the fibrous cap. After the proximal fibrous cap is crossed, distal end 420 of guidewire 414 may encounter tissue, in which case enzyme(s) may be released through a lumen of the catheter. After the tissue is successfully crossed, distal end 420 of guidewire 414 may encounter calcification, in which case chelating agent(s) may be released through a lumen of the catheter. Lastly, a guidewire 414 may encounter a distal fibrous cap and both enzyme(s) and the chelating agent(s), either as separate solutions delivered concurrently or as a single blended solution, may be delivered to effectively weaken the fibrous cap. Accordingly, the composition of occlusion weakening therapy 504 may be changed as needed in an incremental fashion as the CTO is crossed. After occlusion 402 is successfully crossed, catheter shaft 406 may be proximally retracted and withdrawn, while guidewire 414 remains extended through occlusion 402 as shown in FIG. 8 such that a conventional recanalization catheter procedure such as balloon angioplasty and/or stenting may be performed.

One or more enzyme removal features may be incorporated into a catheter used in delivering a occlusion weakening therapy in accordance herewith to remove any excess amount of enzyme(s). In any embodiment described herein, a suction member such as a syringe or vacuum may be attached to the proximal end of the catheter for aspirating excess solution and any debris through a lumen of the catheter. In a single or multi-lumen catheter such as those described herein, aspiration may occur through the same lumen utilized for delivery of the occlusion weakening therapy. Alternatively, the catheter is at least a multi-lumen catheter that includes at least one lumen for delivery of the occlusion weakening therapy and an additional lumen dedicated to aspiration of excess solution and debris. In one embodiment, aspiration of excess solution and debris occurs approximately 10-20 minutes after delivery of the occlusion weakening therapy. Aspiration of excess solution prevents the occlusion weakening therapy from entering a patient's bloodstream. Thus the relatively higher concentrations of enzyme(s) that are utilized in the occlusion weakening therapy in order to speed up the treatment time for weakening the CTO are substantially prevented from entering a patient's bloodstream. In addition, aspiration of debris such as digested tissue that breaks off or separates from the CTO prevents these potentially dangerous fragments from entering a patient's bloodstream.

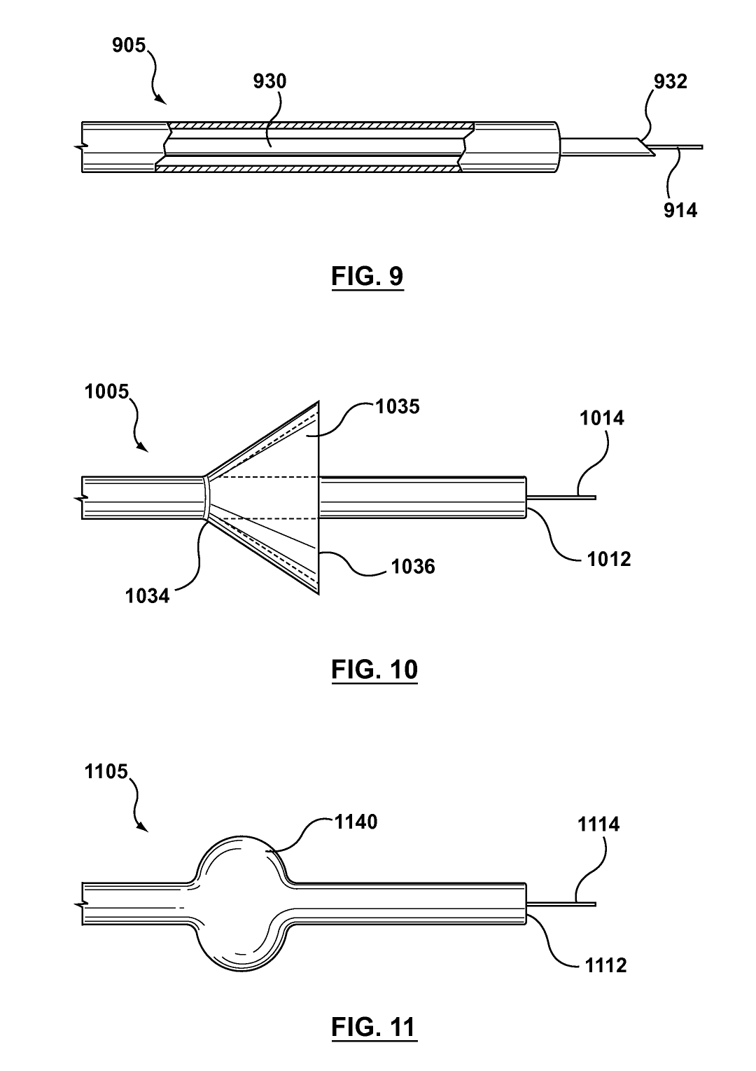

Referring to FIG. 9, another excess enzyme removal feature includes a selectively-extendable needle or cannula 930 operable to penetrate into a CTO and thus guide or direct the occlusion weakening therapy to a CTO. Since the occlusion weakening therapy is directed into a target area of the CTO, the majority of the relatively higher concentrations of enzyme(s) that are utilized in the occlusion weakening therapy in order to speed up the treatment time for weakening a CTO are substantially prevented from entering a patient's bloodstream. A catheter 905 may be a single lumen catheter such as catheter 105, a dual lumen catheter such as catheter 205, or a multi-lumen catheter such as catheter 305. Needle 930 is a tubular member that has a lumen and an open distal end 932. A guidewire 914 slidingly disposed within catheter 905 may be passed through the lumen of needle 930. Needle 930 is moveable back and forth between a retracted position where open distal end 932 is within a lumen of catheter 905 and an extended position wherein a distal portion thereof is advanced out of a distal port of catheter 905. Needle 930 is illustrated in an extended position in FIG. 9. For example, United States Patent Application Pub. No. 2008/0154172 to Mauch, which is hereby incorporated by reference herein in its entirety, describes a catheter device having a selectively-extendable needle tip that may be modified for use herein.

FIG. 10 illustrates a portion of a catheter having another enzyme removal feature. A catheter 1005, which may be a single lumen catheter such as catheter 105, a dual lumen catheter such as catheter 205, or a multi-lumen catheter such as catheter 305, includes an expandable suction cup 1035 mounted around an outside surface thereof at a location proximal to distal catheter tip 1012. Suction cup 1035 is illustrated in an expanded/deployed configuration in FIG. 10. Suction cup 1035 is a generally conical, frusto-conical, or semi-spherical component having a proximal circumferential edge 1034 encircling and attached to an outer surface of catheter 1005 and a distal circumferential edge 1036 that expands against the vessel wall. The diameter of proximal circumferential edge 1034 is approximately the outer diameter of catheter 1005, and the diameter of distal circumferential edge 1036 is approximately the inner diameter of the vessel wall. Suction cup 1035 operates in an umbrella-like fashion in that distal circumferential edge 1036 is collapsible against the outer surface of catheter 1005 during delivery to a target CTO, thereby compressing and/or folding the body of suction cup 1035 against catheter 1005 to minimize the diameter thereof. Once the desired targeting position is achieved, suction cup 1035 is expanded such that distal circumferential edge 1036 flares and contacts the vessel wall proximal to the proximal end of the CTO, thereby creating a shield that blocks blood flow to the CTO. In one embodiment, suction cup 1035 is formed from a self-expanding mesh or frame composed of an alloy material such as Nitinol and covered with a polymeric graft material such as but not limited to Dacron, polytetrafluoroethylene (PTFE), expanded polytetrafluoroethylene (ePTFE), or polyethylene and thus be deployed via a retractable sheath. More particularly, a sheath (not shown) may be provided to surround and contain suction cup 1035 in a contracted or compressed position. The sheath is retracted after catheter 1005 is in position within the target vessel, thus releasing suction cup 1035 to assume its expanded or deployed configuration. Suitable suction mechanisms are described in U.S. Pat. No. 7,736,355 to Itou et al., which is herein incorporated by reference in its entirety. For removal, suction cup 1035 is collapsed by re-advancing the sheath thereover after the procedure is completed. In another embodiment (not shown), suction cup 1035 can be opened and closed in an umbrella-like fashion via connecting the outer diameter to the sheath by flexible struts and changing the position of the central strut attachment.

When expanded in situ, suction cup 1035 assists in fixing or securing the location of a distal tip 1012 of catheter 1005 in close proximity to a CTO while also positioning a guidewire 1014 slidingly received within catheter 1005 towards the center of the proximal end of the CTO such that the guidewire may be utilized to penetrate into the CTO. In addition, the delivered occlusion weakening therapy is essentially trapped between the expanded suction cup 1035 and the CTO during the recanalization procedure. Proximal or upstream blood flow is blocked from mixing with the delivered occlusion weakening therapy by expanded suction cup 1035. Aspiration or suction may be applied through a lumen of catheter 1005 to remove any excess solution and debris to prevent the same from entering a patient's bloodstream, thus creating a suction or vacuum force within the space between the proximal end of the CTO and expanded suction cup 1035. Thus higher concentrations of the enzyme(s) that are utilized in the occlusion weakening therapy in order to speed up the treatment time for weakening a CTO are substantially prevented from entering a patient's bloodstream.

FIG. 11 illustrates a portion of a catheter having another enzyme removal feature. A catheter 1105 includes a centering balloon 1140 located proximal to distal catheter tip 1112. Centering balloon 1140 is an inflatable balloon which is in fluid communication with an inflation lumen (not shown) of catheter 1105. The inflation lumen allows inflation fluid received from an inflation device connected to the proximal end of catheter 1105 to be delivered to centering balloon 1140. Due to the addition of the inflation lumen for expanding centering balloon 1140, catheter 1105 must include at least two lumens and thus may be a dual lumen catheter such as catheter 205 or a multi-lumen catheter such as catheter 305. When inflated, centering balloon 1140 fixes or secures the location of catheter tip 1112 in close proximity to a CTO while also positioning a guidewire 1114 slidingly received catheter 1105 towards the center of the proximal end of the CTO such that the guidewire may be utilized to penetrate into the CTO. In addition, during the recanalization procedure, the delivered occlusion weakening therapy is essentially trapped between the inflated centering balloon 1140 and the CTO. Proximal or upstream blood flow is blocked from mixing with the delivered occlusion weakening therapy by inflated balloon 1140. Aspiration or suction may be applied through a lumen of catheter 1105 to remove any excess solution and debris to prevent the same from entering a patient's bloodstream. Thus higher concentrations of the enzyme(s) that are utilized in the occlusion weakening therapy in order to speed up the treatment time for loosening a CTO are substantially prevented from entering a patient's bloodstream.

The following description relates to specific examples of blended solutions that are operable as occlusion weakening therapy to quickly soften and/or loosen a CTO as described herein. Although the examples are blended solutions having one or more enzyme (s) and a chelating agent, in other embodiments the enyzme(s) and chelating agent(s) may be utilized as separate solutions that are selectively delivered to a CTO depending upon what the make-up of the CTO is when encountered as described above.

### Occlusion Weakening Therapy Formulation Example #1

In one example, a blended solution of enzyme(s) and the chelating agent EDTA may be prepared as described herein. A base buffer of an EDTA solution may be prepared ahead of time. The base buffer of an EDTA solution includes weighing out 0.0606 g of cysteine-HCL and charging it into a 100 ml volumetric flask. 2.915 g of EDTA disodium salt is added to the volumetric flask and the flask is filled to the 100 ml mark. The flask is placed on a stir plate and stirred. The fully dissolved solution is poured into an Erlenmeyer flask and the pH of the solution is adjusted to 6.25. The solution is bubbled with an inert gas, such as argon, to remove oxygen therefrom. The base buffer may be stored in a −20° C. freezer until use. 2 mL of the base EDTA buffer is combined with 2 mL of 10 mg/mL papain solution, an enzyme that breaks up protein. 20 ml of the base EDTA buffer is combined with 30 mg of collagenase I, an enzyme that breaks up protein. A calcium chloride (CaCl₂) solution is also formulated by weighing out 10 g of calcium chloride in a 100 ml Erlenmeyer flask, adding 90 ml of DI water, and dissolving the solution by stirring it at room temperature. The CaCl₂ solution is a salt that enhances or increases the performance of enzymes. 1125 μl of the papain solution/base EDTA buffer, 1125 μl of the collagenase I/base EDTA buffer, and 750 μl of the CaCl₂ solution are combined and vortexed.

### Occlusion Weakening Therapy Formulation Example #2

In another example, a base buffer of an EDTA solution may be prepared ahead of time as described above in Example 1. 2 mL of the base EDTA buffer is combined with 2 mL of 10 mg/mL papain solution, an enzyme that breaks up protein. 20 ml of the base EDTA buffer is combined with 30 mg of collagenase I, an enzyme that breaks up protein. 2.5 ml of the base EDTA buffer is combined with 50 mg of collagenase III solution, an enzyme that breaks up protein. A calcium chloride (CaCl₂) solution is also formulated by weighing out 10 g of calcium chloride in a 100 ml Erlenmeyer flask, adding 90 ml of DI water, and dissolving the solution by stirring it at room temperature. The CaCl₂ solution is a salt that enhances or increases the performance of enzymes. 1125 μl of the papain solution/base EDTA buffer, 1125 μl of the collagenase I/base EDTA buffer, 750 μl of the CaCl₂ solution, and 500 μl of the collagenase III/base EDTA buffer are combined and vortexed.

### Occlusion Weakening Therapy Formulation Example #3

In another example, a base buffer of an EDTA solution may be prepared ahead of time as described above in Example 1. 5 ml of the base EDTA buffer is combined with 100 mg of collagenase I, an enzyme that breaks up protein. A calcium chloride (CaCl₂) solution is also formulated by weighing out 10 g of calcium chloride in a 100 ml Erlenmeyer flask, adding 90 ml of DI water, and dissolving the solution by stirring it at room temperature. The CaCl₂ solution is a salt that enhances or increases the performance of enzymes. 1125 μl of 20 mg/ml papain solution, 1125 μl of the 20 mg/ml collagenase I/base EDTA buffer, and 750 μl of the CaCl₂ solution are combined and vortexed.

### Occlusion Weakening Therapy Formulation Example #4

In another example, a base buffer of an EDTA solution may be prepared ahead of time as described above in Example 1. 5 ml of the base EDTA buffer is combined with 100 mg collagenase I, an enzyme that breaks up protein. 2.5 ml of the base EDTA buffer is combined with 50 mg of collagenase III, an enzyme that breaks up protein. A calcium chloride (CaCl₂) solution is also formulated by weighing out 10 g of calcium chloride in a 100 ml Erlenmeyer flask, adding 90 ml of DI water, and dissolving the solution by stirring it at room temperature. The CaCl₂ solution is a salt that enhances or increases the performance of enzymes. 1125 μl of 20 mg/ml papain solution, 1125 μl of the 20 mg/ml collagenase I/base EDTA buffer, 750 μl of the CaCl₂ solution, and 500 μl of the 20 mg/mL collagenase III solution/base EDTA buffer are combined and vortexed.

### Occlusion Weakening Therapy Formulation Example #5

In another example, a base buffer of an EDTA solution may be prepared ahead of time as described above in Example 1. 2.5 ml of the base EDTA buffer is combined with 50 mg collagenase III, an enzyme that breaks up protein to prepare a 20 mg/mL collagenase III/base EDTA buffer solution.

### Occlusion Weakening Therapy Formulation Example #6

Serrapeptase (CAS #37312-62-2, EC 3.4.24.40) with a known purity is used as received from the manufacturer/supplier. Serrapeptase is an enzyme from silkworms which breaks up proteins found in tissue of a CTO. The enzyme is weighed and dissolved in a sufficient quantity of PBS to make a solution with a concentration of approximately 20 to 25 mg of enzyme/mL of buffer.

While various embodiments according to the present invention have been described above, it should be understood that they have been presented by way of illustration and example only, and not limitation. It will be apparent to persons skilled in the relevant art that various changes in form and detail can be made therein without departing from the spirit and scope of the invention. Thus, the breadth and scope of the present invention should not be limited by any of the above-described exemplary embodiments, but should be defined only in accordance with the appended claims and their equivalents. It will also be understood that each feature of each embodiment discussed herein, and of each reference cited herein, can be used in combination with the features of any other embodiment. All patents and publications discussed herein are incorporated by reference herein in their entirety.

What is claimed is:

1. A method of recanalizing a total occlusion in a body vessel, the method comprising the steps of:
delivering an occlusion weakening therapy effective to soften and/or loosen the occlusion via a catheter located proximally adjacent to the occlusion, wherein the occlusion weakening therapy includes a first therapeutic agent targeted to soften and/or loosen a first material of the occlusion and a second therapeutic agent targeted to soften and/or loosen a second material of the occlusion and wherein the first and second therapeutic agents are selectively deliverable concurrently or consecutively depending on a material composition of the occlusion to be recanalized; and
advancing a distal end of a guidewire into the weakened occlusion until the distal end of the guidewire crosses the occlusion and exits distal thereof.

2. The method of claim 1, wherein the first therapeutic agent is at least one enzyme targeted to loosen tissue which is the first material of the occlusion and the second therapeutic agent is a chelating agent targeted to soften calcification which is the second material of the occlusion.

3. The method of claim 2, wherein the chelating agent is selected from the group consisting of EDTA, EGTA, citric acid, and acetic acid.

4. The method of claim 2, wherein the step of delivering the occlusion weakening therapy includes selectively delivering only the at least one enzyme to loosen the tissue within the occlusion.

5. The method of claim 2, wherein the step of delivering the occlusion weakening therapy includes selectively delivering only the chelating agent to soften the calcification within the occlusion.

6. The method of claim 2, wherein the step of delivering the occlusion weakening therapy includes concurrently delivering both the at least one enzyme and the chelating agent to soften and loosen a fibrous cap of the occlusion that includes both tissue and calcification.

7. The method of claim 1, wherein the catheter includes only one lumen and the first and second therapeutic agents are independently deliverable through the only one lumen or deliverable as a blended solution that includes both the first and second therapeutic agents.

8. The method of claim 1, wherein the catheter includes at least a first lumen for delivering the first therapeutic agent and a second lumen for delivering the second therapeutic agent.

9. The method of claim 1, further comprising:
using ultrasound to determine the material composition of at least a portion of the occlusion to decide whether the first and/or second therapeutic agents is to be delivered to the occlusion.

10. The method of claim 1, wherein the step of delivering the occlusion weakening therapy continues until at least the distal tip of the guidewire distally exits the occlusion.

11. A method of recanalizing a total occlusion in a body vessel, the method comprising the steps of:
positioning distal ends of a catheter and a guidewire proximally adjacent to the occlusion, wherein the guidewire is slidingly received within the catheter;
delivering an occlusion weakening therapy to the occlusion via at least one lumen of the catheter, wherein the occlusion weakening therapy is effective to soften and/or loosen the occlusion in thirty minutes or less after delivery thereof; and
advancing the distal end of the guidewire within the occlusion after the step of delivering the occlusion weakening therapy softens and/or loosens the occlusion.

12. The method of claim 11, wherein the occlusion weakening therapy is effect to soften and/or loosen the occlusion for crossing in five minutes or less after delivery thereof and wherein the step of advancing the distal end of the guidewire within the occlusion is performed at or near the completion of the time period required to soften and/or loosen the occlusion.

13. The method of claim 11, wherein the step of positioning includes extending a needle through the catheter and into the occlusion and the step of delivering the occlusion weakening therapy includes delivering the weakening therapy through the needle.

14. The method of claim 11, wherein the step of positioning includes expanding an expandable suction cup proximate to the distal end of the catheter against a wall of the body vessel to prevent the weakening therapy from entering the bloodstream.

15. The method of claim 11, wherein the step of positioning includes inflating a centering balloon of the catheter against a wall of the body vessel to prevent the occlusion weakening therapy from entering the bloodstream.

16. The method of claim 11, further comprising:
the step of aspirating any excess solution of the occlusion weakening therapy and debris of the occlusion through the catheter after the step of delivering the occlusion weakening therapy.

17. The method of claim 11, wherein the occlusion weakening therapy includes at least one enzyme and a chelating agent.

18. The method of claim 17, wherein the at least one enzyme and the chelating agent are selectively deliverable together or separately depending on a material composition of the portion of the occlusion to be recanalized.

19. The method of claim 17, wherein the step of delivering the occlusion weakening therapy includes delivering the at least one enzyme when tissue within the occlusion is encountered, delivering the chelating agent when calcification within the occlusion is encountered, or delivering both the at least one enzyme and the chelating agent when a fibrous cap of the occlusion is encountered.

20. The method of claim 11, wherein the step of delivering the occlusion weakening therapy continues during advancement of the distal end of the guidewire through the occlusion.
